(19) United States
(12) Patent Application Publication
(10) Pub. No.: US 2014/0228575 A1
(43) Pub. Date: Aug. 14, 2014

(54) Process for the Preparation of Solifenacin and Salts Thereof

(71) Applicants: Jordi Bessa Bellunt, Barcelona (ES); Marina Corbella Morato, Barcelona (ES)

(72) Inventors: Jordi Bessa Bellunt, Barcelona (ES); Marina Corbella Morato, Barcelona (ES)

(73) Assignee: ISOCHEM, Vert le Petit (FR)

(21) Appl. No.: 14/127,572

(22) PCT Filed: Jun. 22, 2011
(86) PCT No.: PCT/EP2011/060398

§ 371 Date: Mar. 6, 2014

(51) Int. Cl. C07D 453/02

(52) U.S. Cl. CPC C07D 453/02
USPC 546/137

(57) ABSTRACT

The invention provides a new process for the preparation of solifenacin or a pharmaceutically acceptable acid addition salt thereof, comprising reacting (R)-quinuclidin-3-yl phenethylcarbamate with benzaldehyde in the presence of an acid to obtain a diasteroisomeric mixture (S,R)—((R)-quinuclidin-3-yl) 1-phenyl-3,4-dihydroisoquinoline-2(1H)-carboxylate of formula (IV) which can be resolved and the solifenacin or a pharmaceutically acceptable acid addition salt thereof recovered. The invention also provides the new key intermediate (R)-quinuclidin-3-yl phenethylcarbamate involved in the process. Further the invention provides a method for the transformation of (R)—((R)-quinuclidin-3-yl) 1-phenyl-3,4-dihydroisoquinoline-2(1H)-carboxylate into a diasteroisomeric mixture (S,R)—((R)-quinuclidin-3-yl) 1-phenyl-3,4-dihydroisoquinoline-2(1H)-carboxylate.

### FIELD OF INVENTION

The present invention relates to an improved process for obtaining (S)—((R)-quinuclidin-3-yl) 1-phenyl-3,4-dihydroisoquinoline-2(1H)-carboxylate, (solifenacin) or an acid addition salt thereof, in particular, a pharmaceutically acceptable acid addition salt thereof. The invention also relates to a new intermediate compound useful for the synthesis of solifenacin. Further the invention relates to a method for the transformation of (R)—((R)-quinuclidin-3-yl) 1-phenyl-3,4-dihydroisoquinoline-2(1H)-carboxylate into a diasteroisomeric mixture (S,R)—((R)-quinuclidin-3-yl) 1-phenyl-3,4-dihydroisoquinoline-2(1H)-carboxylate.

### BACKGROUND OF THE INVENTION

Solifenacin (3R)-1-azabicyclo[2.2.2]oct-3-yl-(1S)-1-phenyl-3,4-dihydroisoquinoline-2-(1H)-carboxylate or 1(S)-phenyl-1,2,3,4-tetrahydroisoquinoline-2-carboxylic acid 3(R)-quinuclidinyl ester, also known as YM-905 (in its free base form) has the following structure:

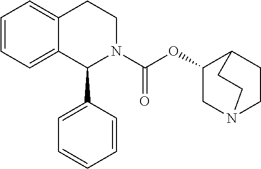 [SMILES: O=C(O[C@H]1CN2CCC1CC2)N1CCc2ccccc2[C@@H]1c1ccccc1]

Solifenacin and its salts are used as therapeutic agents for Pollakiuria and incontinence of urine due to hyperactive bladder.

The drug solifenacin and its pharmaceutically acceptable salts were first reported in EP 0 801 067. The following Scheme 1 shows the synthetic route disclosed therein for the preparation of (1 RS,3′RS)-solifenacin and (1S,3′RS)-solifenacin:

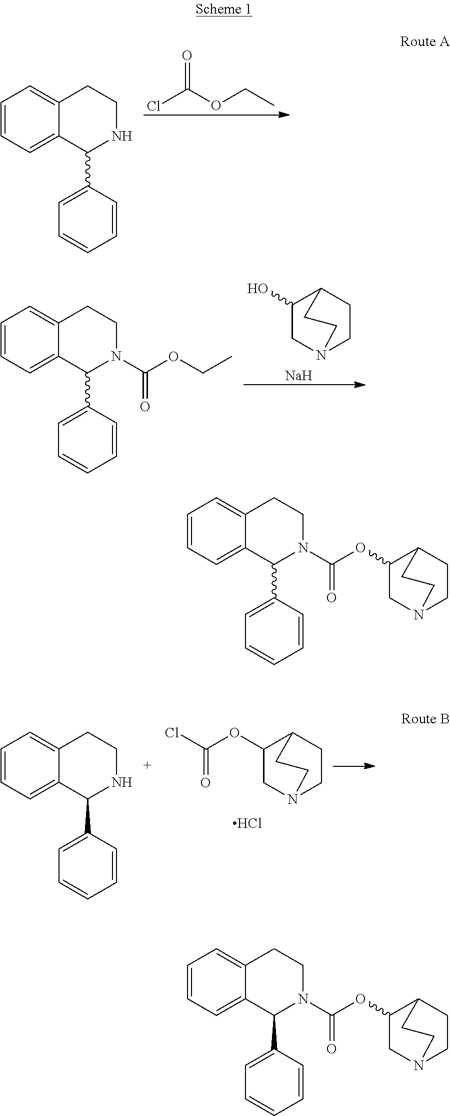

The processes described in EP 0 801 067 are not however very efficient or suitable for industrial scale-up and each route presents several disadvantages. In that sense in route A an excess of compound 3(R)-quinuclidinol of 3 equivalents is needed, strong bases in high concentrations, such as 60% NaH are needed and yields are low. Route B on the other hand requires long reaction times at high temperatures, such as 33 hours at 80° C., the use of toxic solvents such as pyridine or toxic reagents such as phosgene for the preparation of chloroformiates.

Alternative processes have been described in the prior art. Among those processes the following Scheme 2 shows the synthetic route disclosed in WO 2005075474 for the preparation of solifenacin and solifenacin succinate:

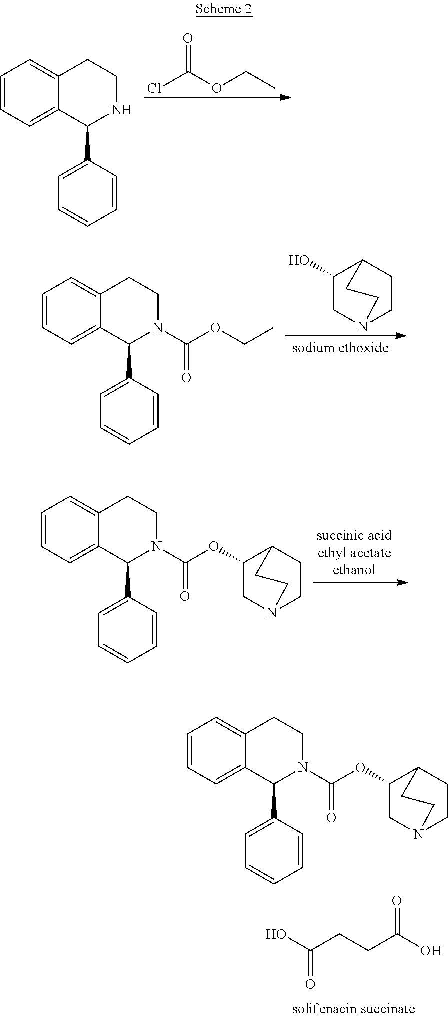

Patent application WO 2008062282 discloses a process comprising reacting the chiral compound (1S)-1-phenyl-1,2,3,4-tetrahydroisoquinoline with a compound of formula: LG-C(O)-LG where LG represents 1H-imidazole-1-yl, 4-methyl-[1,2,4]oxadiazolidine-3,5-dione-2-yl, or 1H-1,2,4-triazol-1-yl or CCl3 to obtain a compound of the following formula:

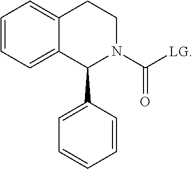

Said compound is further reacted with 3(R)-quinuclidinol, which is activated with a base to form an alkoxide, in the presence of a Lewis acid to give solifenacin.

Patent application WO2008011462 relates to a process for the preparation of solifenacin comprising first the preparation of the chiral compound (1S)-1-phenyl-1,2,3,4-tetrahydroisoquinoline, which is further reacted to obtain a compound of formula (IV), which is then reacted with 3(R)-quinuclidinol of formula (V) in the presence of a base:

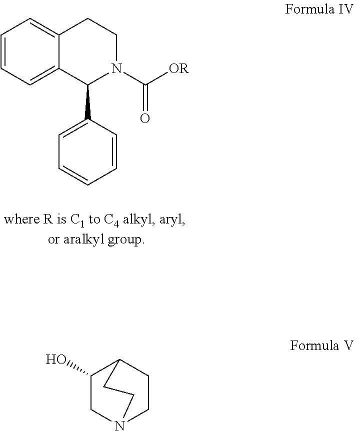

WO2008120080 relates to the following process shown in Scheme 3 which as can be seen involves the use of the chiral compound (1S)-1-phenyl-1,2,3,4-tetrahydroisoquinoline just like all methods previously mentioned:

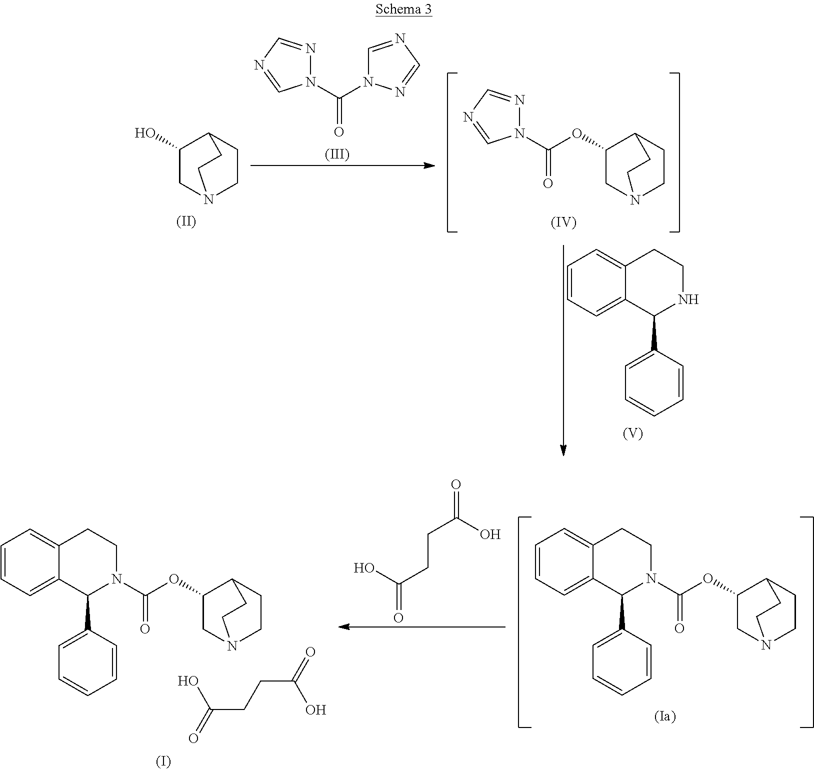

Finally WO 2007076116 discloses a process for preparing solifenacin represented in the following Schema 4 which not only comprises the use of the chiral compound (1S)-1-phenyl-1,2,3,4-tetrahydroisoquinoline, but also the use of intermediates and reagents not easily accessible:

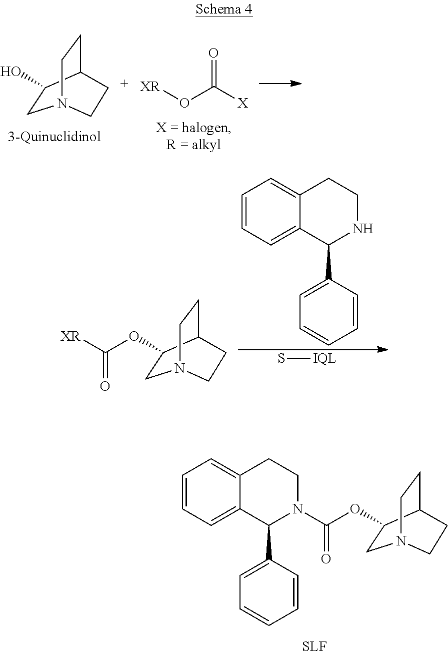

The different synthetic routes of the state of the art suffer from different drawbacks. In particular, all of the above mentioned routes involve the use of the key pure chiral intermediate compound (1S)-phenyl-1,2,3,4-tetrahydroisoquinoline which is costly and difficult to prepare.

Since acid salts of solifenacin are/is being developed as a commercial pharmaceutical product, (solifenacin monosuccinate) it is therefore necessary to solve the problems associated with the processes of the state of the art and to provide an alternative process for obtaining solifenacin which improves the cost of the process using more cost-effective and less hazardous starting materials and reagents, and which is therefore more productive. Said process must advantageously be industrially scaleable and must provide solifenacin with good yield and quality.

### SUMMARY OF THE INVENTION

The present invention is thus faced with the problem of providing an alternative process for obtaining solifenacin or pharmaceutically acceptable salts thereof which overcomes all or at least part of the previously mentioned drawbacks.

The solution provided by the invention is based on the fact that the inventors have surprisingly discovered that it is possible to prepare solifenacin or a pharmaceutically acceptable acid addition salt thereof starting from a new chiral compound, (R)-quinuclidin-3-yl phenethylcarbamate of the following formula (III):

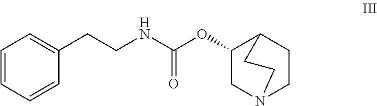 [SMILES: O=C(NCCc1ccccc1)O[C@H]1CN2CCC1CC2]

which is reacted with benzaldehyde in the presence of an acid yielding by cyclization a diastereisomeric mixture of (S,R)—((R)-quinuclidin-3-yl) 1-phenyl-3,4-dihydroisoquinoline-2(1H)-carboxylate of formula (IV)

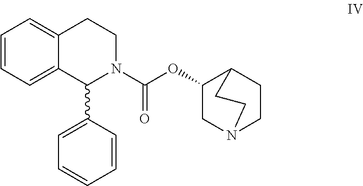 [SMILES: O=C(O[C@H]1CN2CCC1CC2)N1CCc2ccccc2C1c1ccccc1]

which can easily be separated into their diasteroisomers (R)—((R)-quinuclidin-3-yl) 1-phenyl-3,4-dihydroisoquinoline-2(1H)-carboxylate and (S)—((R)-quinuclidin-3-yl) 1-phenyl-3,4-dihydroisoquinoline-2(1H)-carboxylate to recover (S)—((R)-quinuclidin-3-yl) 1-phenyl-3,4-dihydroisoquinoline-2(1H)-carboxylate (solifenacin) of formula (V)

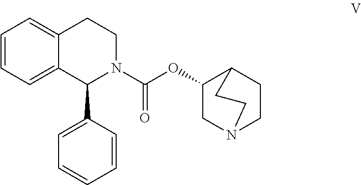 [SMILES: O=C(O[C@H]1CN2CCC1CC2)N1CCc2ccccc2[C@@H]1c1ccccc1]

or an acid addition salt thereof, in particular, a pharmaceutically acceptable acid addition salt thereof.

The process of the invention is advantageous in that the starting compounds and intermediates are either commercially available or readily available. In that sense benzaldehyde is commercially available, and compound (III) is itself obtained by either one of two alternative and different synthetic routes, hereinafter referred to as Method A and Method B (defined below) which in turn comprise the use of commercially available or cost-effective starting compounds such as, 2-phenylethylamine or 3(R)-quinuclidinol.

The process provided by this invention has also the advantages that the chemical reactions occur with short reaction times, under mild reaction conditions and temperatures, which are in general shorter and milder than those required in other processes of the state of the art, and without involving an increase in the number of steps with respect to known processes. In addition the process does not involve the use of expensive and/or hazardous reagents or intermediates, and provides solifenacin or its additions salts with acids, in particular with pharmaceutically acceptable acids, with good yields and pharmaceutical quality. This all contributes to reducing the overall cost of the process, making it commercially interesting and allowing carrying it out to practice on an industrial scale.

Therefore in one aspect the present invention relates to a process for the obtention of solifenacin or a pharmaceutically acceptable acid addition salt thereof from a compound of formula (III) by reaction with benzaldehyde in the presence of an acid, to yield by cyclization a diastereisomeric mixture of (S,R)—((R)-quinuclidin-3-yl) 1-phenyl-3,4-dihydroisoquinoline-2(1H)-carboxylate which can easily be separated into their diasteroisomers to recover the therapeutically active diasteroisomer (S)—((R)-quinuclidin-3-yl) 1-phenyl-3,4-dihydroisoquinoline-2(1H)-carboxylate (solifenacin) or a pharmaceutically acceptable acid addition salt thereof.

In a further aspect the invention relates to (R)-quinuclidin-3-yl phenethylcarbamate of formula (III), or a salt thereof, useful in the synthetic preparation of solifenacin or a pharmaceutically acceptable acid addition salt thereof.

In still a further aspect the invention relates to a method for (R)—((R)-quinuclidin-3-yl) 1-phenyl-3,4-dihydroisoquinoline-2(1H)-carboxylate into a diasteroisomeric mixture, (S,R)—((R)-quinuclidin-3-yl) 1-phenyl-3,4-dihydroisoquinoline-2(1H)-carboxylate (IV) which comprises the following steps:
treating (R)—((R)-quinuclidin-3-yl) 1-phenyl-3,4-dihydroisoquinoline-2(1H)-carboxylate, or a diasteroisomeric mixture enriched with (R)—((R)-quinuclidin-3-yl) 1-phenyl-3,4-dihydroisoquinoline-2(1H)-carboxylate or an acid addition salt thereof, with a strong acid, and
recovering the resulting diasteroisomeric mixture (S,R)—((R)-quinuclidin-3-yl) 1-phenyl-3,4-dihydroisoquinoline-2(1H)-carboxylate (IV).

The method optionally further comprises the separation of the diasteroisomeric mixture (S,R)—((R)-quinuclidin-3-yl) 1-phenyl-3,4-dihydroisoquinoline-2(1H)-carboxylate into its diastereoisomers.

### DETAILED DESCRIPTION OF THE INVENTION

In one aspect the present invention relates to a process for preparing solifenacin or an acid addition salt thereof, in particular, a pharmaceutically acceptable acid addition salt thereof, from a compound of formula (III) by reaction with benzaldehyde in the presence of an acid, to yield by cyclization a diastereisomeric mixture of (S,R)—((R)-quinuclidin-3-yl) 1-phenyl-3,4-dihydroisoquinoline-2(1H)-carboxylate which can easily be separated into their diasteroisomers to recover the therapeutically active diasteroisomer (S)—((R)-quinuclidin-3-yl) 1-phenyl-3,4-dihydroisoquinoline-2(1H)-carboxylate (solifenacin) or an acid addition salt thereof, in particular, a pharmaceutically acceptable acid addition salt thereof.

The process hereinafter referred to as the process of the invention, is represented in Schema 1.

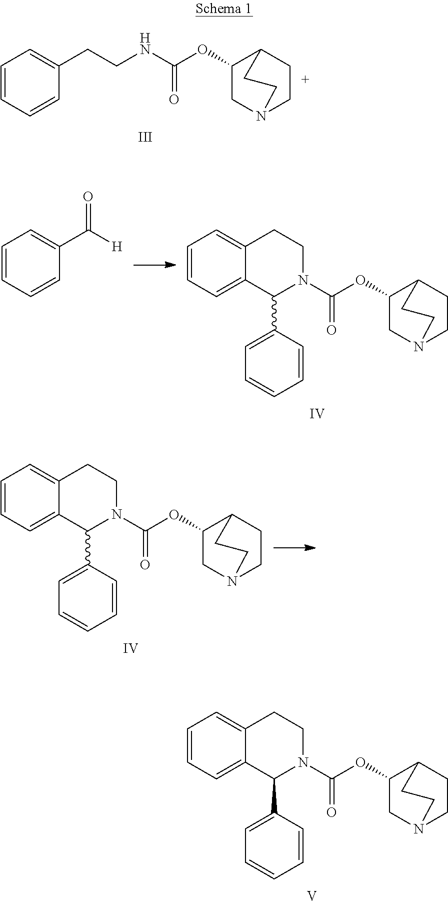

Compound (III) is reacted with benzaldehyde in the presence of an acid which can virtually be any acid selected from the group consisting of organic acids, inorganic acids, Lewis acids and their mixtures. Illustrative non limiting examples of acids are sulfuric acid, acetic acid, methanesulfonic acid, boron trifluoride, titanium (IV) chloride zinc chloride, or combinations thereof. The process is typically carried out by adding first said acid to a cooled mixture of compound (III) and benzaldehyde, and controlling the reaction temperature to be comprised between 0 and 40° C., preferably between 25-30° C. The cyclization reaction yields a diasteroisomeric mixture (S,R)—((R)-quinuclidin-3-yl) 1-phenyl-3,4-dihydroisoquinoline-2(1H)-carboxylate of formula (IV). In the context of the present invention diasteroisomeric mixture of formula (IV) refers to a mixture of (R)—((R)-quinuclidin-3-yl) 1-phenyl-3,4-dihydroisoquinoline-2(1H)-carboxylate and (S)—((R)-quinuclidin-3-yl) 1-phenyl-3,4-dihydroisoquinoline-2(1H)-carboxylate in an about 1:1 ratio. In the context of the present invention an about 1:1 ratio, refers to ratios comprised between 55:45 and 45:55.

The process of the invention comprises further the separation of the diasteroisomers from the diasteroisomeric mixture (S,R)—((R)-quinuclidin-3-yl) 1-phenyl-3,4-dihydroisoquinoline-2(1H)-carboxylate of formula (IV) to recover the desired (S)—((R)-quinuclidin-3-yl) 1-phenyl-3,4-dihydroisoquinoline-2(1H)-carboxylate or solifenacin of formula (V) or an acid addition salt thereof, in particular, a pharmaceutically acceptable acid addition salt thereof.

Separation of the diasteroisomers to recover solifenacin or an acid addition salt thereof can be carried out by any conventional method of resolution of diasteroisomers for example by means of fractional crystallization or conventional chromatographic methods.

According to a particular embodiment the method used is a fractional crystallization method which comprises the following steps:
(i) treating the diasteroisomeric mixture obtained from the reaction of (R)-quinuclidin-3-yl phenethylcarbamate of formula (III) with benzaldehyde in a solvent with an organic or inorganic acid,
(ii) separating a first precipitate enriched in the addition salt of (S)—((R)-quinuclidin-3-yl) 1-phenyl-3,4-dihydroisoquinoline-2(1H)-carboxylate with said acid from the mother liquor enriched in the addition salt of (R)—((R)-quinuclidin-3-yl) 1-phenyl-3,4-dihydroisoquinoline-2(1H)-carboxylate with said acid;
(iii) recovering of the addition salt of (S)—((R)-quinuclidin-3-yl) 1-phenyl-3,4-dihydroisoquinoline-2(1H)-carboxylate with said acid.

The fractional crystallization method can be carried out with any suitable organic or inorganic acid provided that the resulting diasteroisomeric salts present different solubilities in the said solvent to permit separation. In a particular embodiment the resulting addition salt of (S)—((R)-quinuclidin-3-yl) 1-phenyl-3,4-dihydroisoquinoline-2(1H)-carboxylate with said acid presents less solubility in the said solvent than the addition salt of (R)—((R)-quinuclidin-3-yl) 1-phenyl-3,4-dihydroisoquinoline-2(1H)-carboxylate with said acid allowing the formation of a precipitate enriched in the (S)—((R)-quinuclidin-3-yl) 1-phenyl-3,4-dihydroisoquinoline-2(1H)-carboxylate diasteroisomer. Illustrative non limiting examples of acids useful in the crystallization method are succinic acid, oxalic acid and sulphuric acid. After the first precipitate is recovered, the same can be further dissolved in said solvent and can be submitted to one or more further fractional crystallization methods as disclosed above until the addition salt of (S)—((R)-quinuclidin-3-yl) 1-phenyl-3,4-dihydroisoquinoline-2(1H)-carboxylate with said acid is recovered with the desired degree of purity.

Suitable solvents to be used in the crystallization method are for instance ketones such as acetone, methyl ethyl ketone, methyl isopropyl ketone, esters such as ethyl acetate, methyl acetate, alcohols such as ethanol, isopropanol, ethers such as diethyl ether, diisopropylether, and mixtures thereof.

The diasteroisomeric mixture (S,R)—((R)-quinuclidin-3-yl) 1-phenyl-3,4-dihydroisoquinoline-2(1H)-carboxylate of formula (IV) is typically first dissolved in said solvent or mixture of solvents, with heat if convenient; the resulting solution is then cooled at a temperature of typically between 10 and 30° C., and optionally, the solution is seeded with the corresponding acid addition salt of (S)—((R)-quinuclidin-3-yl) 1-phenyl-3,4-dihydroisoquinoline-2(1H)-carboxylate to induce precipitation.

According to a preferred embodiment the fractional crystallization method further comprises the following steps:
a) treating the mother liquor enriched in the addition salt of (R)—((R)-quinuclidin-3-yl) 1-phenyl-3,4-dihydroisoquinoline-2(1H)-carboxylate with said acid, with a base or a second acid,
b) recovering the resulting diasteroisomeric mixture (S,R)—((R)-quinuclidin-3-yl) 1-phenyl-3,4-dihydroisoquinoline-2(1H)-carboxylate and optionally further carrying out the following steps of a fractional crystallization method:
   (i) treating said diasteroisomeric mixture with an organic or inorganic acid;
   (ii) separating a second precipitate enriched in the addition salt of (S)—((R)-quinuclidin-3-yl) 1-phenyl-3,4-dihydroisoquinoline-2(1H)-carboxylate with said acid from the mother liquor enriched in the addition salt of (R)—((R)-quinuclidin-3-yl) 1-phenyl-3,4-dihydroisoquinoline-2(1H)-carboxylate with said acid and
   (iii) recovering of the addition salt of (S)—((R)-quinuclidin-3-yl) 1-phenyl-3,4-dihydroisoquinoline-2(1H)-carboxylate with said acid.

One or more of the steps of the fractional crystallization method can be repeated in a conventional manner until substantially pure, or in any desired degree of purity, therapeutically active solifenacin is recovered.

Solifenacin resulting from the fractional crystallization method is recovered as an acid addition salt, which can be further if desired, be released into its free base and further be transformed into a different acid addition salt, in particular a pharmaceutically acceptable addition salt.

According to a particular embodiment the base used for treating the mother liquor enriched in the addition salt of (R)—((R)-quinuclidin-3-yl) 1-phenyl-3,4-dihydroisoquinoline-2(1H)-carboxylate with said acid, is a suitable conventional strong base. Illustrative non limiting examples are potassium tert-butoxide, sodium hydride, lithium bis(trimethylsilyl)amide and potassium lithium bis(trimethylsilyl)amide.

The inventors however have surprisingly found that is also possible to treat the mother liquor enriched in the addition salt of (R)—((R)-quinuclidin-3-yl) 1-phenyl-3,4-dihydroisoquinoline-2(1H)-carboxylate with said acid, with a second acid to obtain a diasteroisomeric mixture (S,R)—((R)-quinuclidin-3-yl) 1-phenyl-3,4-dihydroisoquinoline-2(1H)-carboxylate. Therefore according to a particular embodiment the second acid used for treating the mother liquor is a suitable strong acid, organic or inorganic, such as methanesulfonic acid, sulfuric acid and aqueous concentrated chlorhydric acid.

The process of the present invention also comprises the preparation of (R)-quinuclidin-3-yl phenethylcarbamate of formula (III), or a salt thereof, a new quiral intermediate compound useful for the preparation of solifenacin or a pharmaceutically acceptable acid addition salt thereof which constitutes a further aspect of the present invention as previously mentioned.

Said starting material of formula (III) is advantageously prepared from commercially available or cost-effective chemical products according to two different alternative synthesis routes, hereinafter referred to as Method A and Method B.

Method A is represented in the following Schema 2:

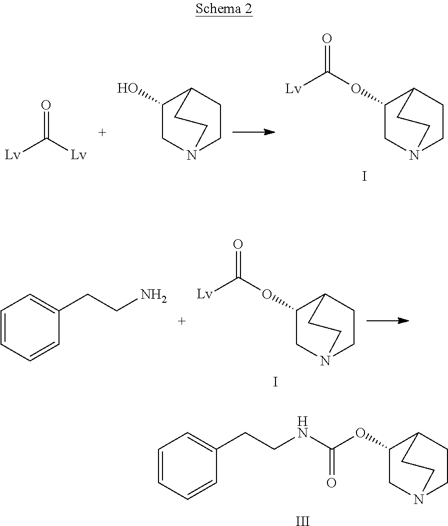

As used in the present invention Lv represents a “leaving group”, which term includes any leaving group well known to the skilled person in the art. Although practically any leaving group can be used in the chemical reactions of Methods A and B illustrative non limiting examples of Lv are —OR group, wherein R represents a linear or branched C1-C6 alkyl group or an aryl group, optionally substituted, an 1H-imidazole-1-yl, 1H-1,2,4-triazol-1-yl, Cl or CCl₃

According to Method A, the process of the invention further comprises the obtention of compound (I) by reacting a compound of formula Lv-C(O)-Lv, where Lv, the same or different, represents a leaving group, with 3(R)-quinuclidinol in a solvent. Reaction is carried out at temperature comprised between −10° C. and 80° C., preferably comprised between −10 and 40° C., more preferably at 0° C. Resulting compound (I) may be purified if desired to be used in the subsequent preparation of compound (III) or the resulting reaction mixture may be subsequently used without need of purification of compound (I).

Compound (III) is prepared by reacting 2-phenethylamine with said compound of formula (I) wherein Lv represents a leaving group, in a solvent, optionally in the presence of a base. Suitable bases for use are for instance amines, more particularly trialkylamines, such as trimethylamine or triethylamine. Reaction temperature is preferably comprised between 0° C. and 30° C. Examples of suitable solvents for use in both reactions of Method A are organic aprotic solvents for instance ethers such as tetrahydrofuran, 1,4-dioxane, esters such as ethyl acetate, isopropyl acetate, toluene, dichloromethane, dimethylformamide and the like, or mixtures thereof. The reactions are conveniently carried out under inert atmosphere. According to a particular embodiment compound of formula Lv-C(O)-Lv is reacted with 3(R)-quinuclidinol and to the resulting reaction mixture 2-phenetylamine is added at a temperature typically comprised between −10° C. and 10° C., preferably at 0° C. The reaction mixture is then allowed to reach room temperature and compound (III) is obtained. Compound (III) can be further used in the process of the invention without need of purification if so desired.

Method B is represented in the following Schema 3:

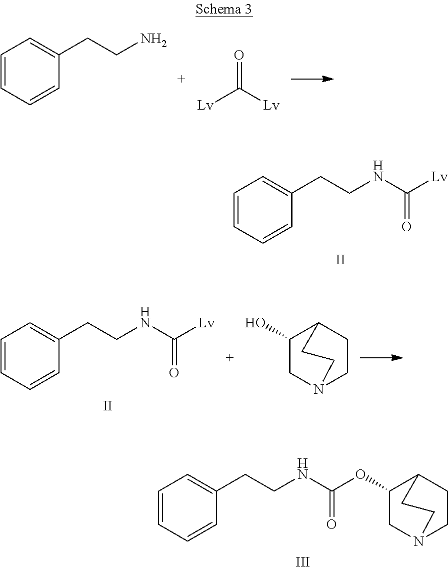

According to the alternative Method B of the process of the invention compound (II) is prepared by reacting 2-phenethylamine with a compound of formula Lv-C(O)-Lv where Lv, the same or different, represents a leaving group in a solvent, optionally in the presence of a base. Suitable bases for use are amines, for instance trialkylamines, such as diisopropylethylamine or triethylamine. Reaction temperature is typically comprised between 0° C. and 80° C., preferably between 0° C. and 40° C. Examples of suitable solvents are organic aprotic solvents for instance, ethers such as tetrahydrofuran, 1,4-dioxane, esters such as ethyl acetate, isopropyl acetate, toluene, dichloromethane, dimethylformamide or mixtures and the like. The resulting compound (II) can be used in the preparation of compound (III) without further purification if desired.

Compound (III) is prepared by reacting compound of formula (II) wherein Lv represents a leaving group as previously defined with 3(R)-quinuclidinol in the presence of a base in a solvent. Examples of suitable solvents are organic aprotic solvents like ethers such as tetrahydrofuran, 1,4-dioxane, esters such as ethyl acetate, isopropyl acetate, toluene, dichloromethane, dimethylformamide or mixtures and the like.

According to a particular embodiment when Lv is —OR, the base activates the 3(R)-quinuclidinol to form the corresponding alkoxide which subsequently reacts with compound (II). The base can virtually be any base capable of activating the 3(R)-quinuclidinol. In a more particular embodiment the base is a metal alkoxide such as sodium methanolate, sodium ethanolate, potassium tert-butoxide or sodium hydride. When such a base is used the reaction takes place optionally under distillation of the generated alcohol such as methanol or ethanol.

Method B is conveniently carried out under inert atmosphere.

Compound (III) can be further used in the process of the invention without purification if so desired.

In still a further aspect the present invention relates to a method for the transformation of (R)—((R)-quinuclidin-3-yl) 1-phenyl-3,4-dihydroisoquinoline-2(1H)-carboxylate into a diasteroisomeric mixture, (S,R)—((R)-quinuclidin-3-yl) 1-phenyl-3,4-dihydroisoquinoline-2(1H)-carboxylate (IV) which comprises the following steps:
treating (R)—((R)-quinuclidin-3-yl) 1-phenyl-3,4-dihydroisoquinoline-2(1H)-carboxylate or a diasteroisomeric mixture enriched with (R)—((R)-quinuclidin-3-yl) 1-phenyl-3,4-dihydroisoquinoline-2(1H)-carboxylate or an acid addition salt thereof with a strong acid, and
recovering the resulting diasteroisomeric mixture (S,R)—((R)-quinuclidin-3-yl) 1-phenyl-3,4-dihydroisoquinoline-2(1H)-carboxylate (IV).

According to a particular embodiment the starting compound (R)—((R)-quinuclidin-3-yl) 1-phenyl-3,4-dihydroisoquinoline-2(1H)-carboxylate is a pure diasteroisomer or a acid addition salt thereof. In another particular embodiment the starting compound is a diasteroisomeric mixture, or a acid addition salt thereof, enriched with (R)—((R)-quinuclidin-3-yl) 1-phenyl-3,4-dihydroisoquinoline-2(1H)-carboxylate presenting a d.e in (R)—((R)-quinuclidin-3-yl) 1-phenyl-3,4-dihydroisoquinoline-2(1H)-carboxylate of more than 5%. The resulting diasteroisomeric mixture of the method presents a d.e in (R)—((R)-quinuclidin-3-yl) 1-phenyl-3,4-dihydroisoquinoline-2(1H)-carboxylate of 5% or less.

In another particular embodiment the starting compound is any (R)—((R)-quinuclidin-3-yl) 1-phenyl-3,4-dihydroisoquinoline-2(1H)-carboxylate or diasteroisomeric mixture enriched with (R)—((R)-quinuclidin-3-yl) 1-phenyl-3,4-dihydroisoquinoline-2(1H)-carboxylate obtained according to any synthetic route for the preparation of solifenacin or acid addition salt thereof. Accordingly in still one particular embodiment a mother liquor enriched with (R)—((R)-quinuclidin-3-yl) 1-phenyl-3,4-dihydroisoquinoline-2(1H)-carboxylate resulting from a fractional crystallization method for the separation of a diasteroisomeric mixture (S,R)—((R)-quinuclidin-3-yl) 1-phenyl-3,4-dihydroisoquinoline-2(1H)-carboxylate, constitutes the starting compound in the method of transformation of the invention. In a more particular embodiment said mother liquor is obtained in the process of the present invention above disclosed.

The strong acid used in the method of transformation of the invention is virtually any organic or inorganic strong acid. Non limiting examples are methanesulfonic acid, sulfuric acid, aqueous concentrated chlorhydric acid or mixtures thereof. The acid is used in an amount of 1 or more equivalents, preferably in an amount of between 5 to 25 equivalents. When used in excess the acid also acts as a solvent.

The method is typically carried out at a temperature comprised between 0° C. and 50° C., preferably between 15-30° C., more preferably between 20-25° C., and even more preferably under inert atmosphere.

The method for the transformation of the invention optionally further comprises the separation of the recovered diasteroisomeric mixture (S,R)—((R)-quinuclidin-3-yl) 1-phenyl-3,4-dihydroisoquinoline-2(1H)-carboxylate into its diastereoisomers according to well known methods from the state of the art such as fractional crystallization, as above in detail disclosed, or conventional chromatographic methods.

The method of transformation of the invention presents the important advantage that in any synthetic route for the preparation of solifenacin leading to mixtures of (S,R)—((R)-quinuclidin-3-yl) 1-phenyl-3,4-dihydroisoquinoline-2(1H)-carboxylate, the method can readily and easily be carried out to transform the undesired diasteroisomer (R)—((R)-quinuclidin-3-yl) 1-phenyl-3,4-dihydroisoquinoline-2(1H)-carboxylate in the therapeutically active (S)—((R)-quinuclidin-3-yl) 1-phenyl-3,4-dihydroisoquinoline-2(1H)-carboxylate (solifenacin).

The foregoing is illustrative of the present invention. This invention however is not limited to the following precise embodiments described herein, but encompasses all equivalent modifications within the scope of the claims which follow.

### EXAMPLES

### Example 1

### Preparation of (R)-quinuclidin-3-yl phenethylcarbamate (compound III), Method A

14 g (86.17 mmol) of 1,1′-carbonyldiimidazole were added under nitrogen atmosphere to a suspension of 10 g (78.51 mmol) of 3(R)-quinuclidinol in 150 mL of THF at 0° C. The reaction was left under stirring at 0° C. for 4 h until total conversion to (R)-imidazole-1-carboxylic acid 1-azabicyclo[2.2.2]oct-3-yl ester (compound Ia) was observed by TLC (CH₂Cl₂:MeOH:aqNH₃ 9:1:0.1). To the obtained solution was added dropwise at 0° C. a mixture of 9.9 mL (78.51 mmol) of 2-phenethylamine and 10.0 mL (78.51 mmol) of triethylamine. After 30 min at 0° C. the reaction was allowed to reach room temperature and was left stirring under nitrogen atmosphere over night. The solvent was distilled under vacuum and the residue was dissolved in 100 mL of dichloromethane and extracted twice with 50 mL of 1N HCl. The aqueous extracts were basified to pH 10 with potassium carbonate and the solid obtained was collected by filtration and dried to obtain 14.07 g (65.2%) of (R)-quinuclidin-3-yl phenethylcarbamate.

RMN 1H (CDCl3), δ(ppm): 1.2-1.9 (m, 4H, 2×CH2); 1.9-2.1 (m, 1H, CH); 2.5-3.0 (m, 7H, 2×CH2-N+CH2-Ar+½ CH2-N); 3.1-3.3 (dd, 1H, ½ CH2-N); 3.3-3.6 (m, 2H, CH2-NH); 4.5-4.9 (m, 2H, NH+CH—O); 7.1-7.4 (m, 5H, Ar).

### Example 2

### Preparation of (R)-quinuclidin-3-yl phenethylcarbamate (compound III), Method B

### a) Preparation of Ethyl Phenethylcarbamate (Compound IIa).

At 0° C., 13.9 mL (145 mmol) of ethyl chloroformate were added dropwise to a solution of 14.72 g (145 mmol) of triethylamine and 15.99 g (132 mmol) of 2-phenethylamine in 300 mL of dichloromethane. After stirring the mixture at room temperature for 3 hours, it was washed successively with water, HCl 1M and brine, and evaporated to dryness under a reduced pressure. Crude was obtained as a pale yellow oil (27.77 g) and used in the next step without further purification.

RMN 1H (CDCl3), δ(ppm): 1.26 (t, 3H, CH3); 2.84 (t, 2H, CH2); 3.3-3.5 (m, 2H, CH2-N); 4.13 (q, 2H, CH2-0); 4.72 (s, 1H, NH); 7.1-7.4 (m, 5H, Ar).

b) Preparation of (R)-quinuclidin-3-yl phenethylcarbamate (compound iii).

To a solution of 1.33 g (7.56 mmol) of crude ethyl phenethylcarbamate in a mixture of 1 mL of DMF and 20 mL of toluene were added 1.01 g (7.56 mmol) of 3(R)-quinuclidinol. The mixture was heated, and after the starting substances had dissolved completely, 0.27 mL (1.26 mmol) of sodium methoxide 25% in methanol were added. While the reaction proceeded, the azeotropic toluene-ethanol mixture was distilled off. After 4 hours, the mixture was cooled to 20-25° C. and 20 mL of water were added. The aqueous layer was separated and the organic layer was extracted twice with 20 mL of HCl 1M. Potassium carbonate was added to the combined aqueous phases and the pH was adjusted to 10. After cooling to 0° C. the precipitated solid was collected by filtration, washed with 10 mL of water and then dried under a reduced pressure to obtain (R)-quinuclidin-3-yl-phenethylcarbamate as a pale orange solid (0.7 g, 40%).

### Example 3

### Preparation of (R)-quinuclidin-3-yl phenethylcarbamate (compound III), Method B

a) Preparation of N-phenethyl-1H-imidazole-1-carboxamide (compound IIIb).

5.0 g (30.8 mmol) of 1,1′-carbonyldiimidazole were added under nitrogen atmosphere to a solution of 3.13 g (25.8 mmol) of 2-phenethylamine in 70 mL of dichloromethane. After stirring the mixture at room temperature for 3.5 hours 70 mL of water were added. The aqueous layer was separated, the organic layer was washed with 70 mL of water and evaporated to dryness under reduced pressure. 5.11 g (92%) of N-phenethyl-1H-imidazole-1-carboxamide was obtained as a white solid and used in the next step without further purification.

RMN 1H (CDCl3), δ(ppm): 3.00 (t, 2H, CH2Ar); 3.72 (m, 2H, CH2N); 6.50 (s, 1H, NH); 7.08 (s, imidazole); 7.00-7.45 (m, 6H, Ar+ imidazole); 8.28 (s, 1H, imidazole).
b) At room temperature, 2.36 g (18.6 mmol) of 3(R)-quinuclidinol were slowly added under nitrogen atmosphere to a suspension of 0.82 g (20.5 mmol) of 60% sodium hydride in 40 mL of dry tetrahydrofuran. The system was left stirring at room temperature, and after the hydrogen evolution had stopped, 4.0 g (18.6 mmol) of N-phenethyl-1H-imidazole-1-carboxamide were added. After 4 hours, a volume of 40 mL of HCl 1N was added and the mixture was stirred for 10 minutes. The organic layer was allowed to separate in a separating funnel and was further extracted twice with 40 mL of HCl 1N. The combined aqueous extracts were basified with potassium carbonate to pH 10, cooled to 0° C. and the precipitated solid was collected by filtration, washed with 10 mL of water and then dried under a reduced pressure to obtain 3.17 g (63.5%) of (R)-quinuclidin-3-yl-phenethylcarbamate as a white solid.

### Example 4

### Preparation of (S,R)—((R)-quinuclidin-3-yl) 1-phenyl-3,4-dihydroisoquinoline-2(1H)-carboxylate (compound IV)

70 mL of methanosulfonic acid were added dropwise to an ice bath cooled mixture of 12 g (43.74 mmol) of (R)-quinuclidin-3-yl-phenethylcarbamate and 8.9 mL (87.64 mmol) of benzaldehyde controlling the temperature at 25-30° C. The reaction was left stirring 4 h at room temperature until complete disappearance of (R)-quinuclidin-3-yl-phenethylcarbamate is observed by TLC (CH₂Cl₂:MeOH:aqNH₃ 9:1:0.1). After slowly adding 60 mL of water at 20-25° C., the aqueous solution was washed twice with 100 mL of heptane, extracted four times with 100 mL of dichloromethane and the combined organic phases were treated with 100 mL of water and the pH adjusted to 10 with potassium carbonate. The aqueous phase was separated and the organic phase was concentrated to dryness under reduced pressure to obtain 13.47 g (85%) of (S,R)—((R)-quinuclidin-3-yl) 1-phenyl-3,4-dihydroisoquinoline-2(1H)-carboxylate as a yellowish oil.

RMN 1H (CDCl3): 1.30-1.95 (m, 4H); 2.00-2.15 (m, 1H); 2.60-3.10 (m, 7H); 3.20-3.45 (m, 2H); 4.05 (bs, 1H); 4.70-4.90 (m, 1H); 6.10-6.60 (m, 1H); 7.00-7.35 (m, 9H).

### Example 5

### Preparation of (S,R)—((R)-quinuclidin-3-yl) 1-phenyl-3,4-dihydroisoquinoline-2(1H)-carbon/late (compound IV)

A mixture of 40 mL of acetic acid and 40 mL of sulfuric acid was added dropwise to an ice bath cooled mixture of 14 g (51.03 mmol) of (R)-quinuclidin-3-yl-phenethylcarbamate and 7.8 mL (76.80 mmol) of benzaldehyde controlling the temperature at 25-30° C. The reaction was left stirring 4 h at room temperature until complete disappearance of (R)-quinuclidin-3-yl-phenethylcarbamate is observed by TLC (CH₂Cl₂:MeOH:aqNH₃ 9:1:0.1). The resultant mixture was slowly added to 80 mL of water at 0° C. and extracted twice with 80 mL of dichloromethanethe. The organic phase was distilled to dryness under reduced pressure and the residue solved in 50 mL of water, washed three times with 50 mL of toluene and, after adjusting the pH of the aqueous phase to 10 with potassium carbonate, extracted twice with 50 mL of toluene. The organic phase was concentrated to dryness under reduced pressure to obtain 16.5 g (89%) of (S,R)—((R)-quinuclidin-3-yl) 1-phenyl-3,4-dihydroisoquinoline-2(1H)-carboxylate as a yellowish oil.

### Example 6

### Preparation of solifenacin monosuccinate

1.47 g (12.45 mmol) of succinic acid were added to a solution of 4.02 g (11.09 mmol) of (S,R)—((R)-quinuclidin-3-yl) 1-phenyl-3,4-dihydroisoquinoline-2(1H)-carboxylate in a mixture of 15.5 mL of ethyl acetate and 6.8 mL of ethanol. The suspension was heated to assure complete solution and after cooling to 20-25° C. was seeded with solifenacin succinate and left stirring at room temperature for 4 h. The solid was recovered by filtration and washed with 5 mL of ethyl acetate to obtain 1 g (18.8%, d.e.=99.0%) of solifenacin monosuccinate.

A second crop of 0.70 g (13.2%, d.e.=98.3%) of solifenacin monosuccinate was obtained from the filtrate by vacuum distillation of solvents, recovering of solifenacin base in the organic phase with toluene and 10% aqueous solution of potassium carbonate, epimerization of (R,R) enriched mixture of diastereoisomers of solifenacin using 14 mL (14 mmol) of 1M solution of lithium bis(trimethylsilyl)amide in toluene and final resolution with 0.87 g (7.36 mmol) of succinic acid in a mixture of 8.3 mL of ethyl acetate and 1.66 mL of ethanol.

### Example 7

### Preparation of solifenacin monooxalate

0.49 g (1.35 mmol) of (S,R)—((R)-quinuclidin-3-yl) 1-phenyl-3,4-dihydroisoquinoline-2(1H)-carboxylate and 0.12 g (1.33 mmol) of oxalic acid were completely dissolved in 5 mL of ethanol and concentrated to dryness under reduced pressure. The residue was heated to 50° C. in a mixture of 2 mL of isopropanol and 0.5 mL of diisopropyl ether and the solution was seeded with solifenacin monooxalate. After allowing the suspension to cool to 30° C. over 1 h, the solid was collected by filtration to obtain after drying 0.05 g (8.2%, d.e.=93.6%) of solifenacin monooxalate. The filtrate was left to stand over night at room temperature and a second crop of 0.10 g (16.4%, d.e.=70.4%) of solifenacin monooxalate was obtained.

### Example 8

### Preparation of Solifenacin Hidrogensulfate

0.095 g (0.97 mmol) of sulfuric acid were added to a solution of 0.35 g (0.96 mmol) of (S,R)—((R)-quinuclidin-3-yl) 1-phenyl-3,4-dihydroisoquinoline-2(1H)-carboxylate in 1.5 mL of ethanol. 2 mL of ethyl acetate were added and the mixture was heated to 50° C. to obtain a complete solution. After allowing to cool to 0° C., the mixture was seeded with solifenacin hydrogensulfate and left stirring at 0° C. for 1 h. The solid was collected by filtration to obtain after drying 0.05 g (11.4%, d.e.=88.2%) of solifenacin hydrogensulfate.

### Example 9

### Recovering of (S,R)—((R)-quinuclidin-3-yl) 1-phenyl-3,4-dihydroisoquinoline-2(1H)-carboxylate (compound IV)

0.50 g (1.37 mmol) of diastereoisomeric mixture enriched with isomer (R)—((R)-quinuclidin-3-yl) 1-phenyl-3,4-dihydroisoquinoline-2(1H)-carboxylate (d.e.=24.0%) was treated with 2 mL of methanesulfonic acid at 20-25° C. over 16 h under stirring and nitrogen atmosphere. The reaction was poured over 4 ml of water, washed twice with 2 mL of toluene and extracted twice with 2 mL of dichloromethane. After removing the solvent at reduced pressure it was obtained a mixture of (S,R)—((R)-quinuclidin-3-yl) 1-phenyl-3,4-dihydroisoquinoline-2(1H)-carboxylate as methanesulfonate salt (d.e.=4.0%).

### Example 10

### Recovering of (S,R)—((R)-quinuclidin-3-yl) 1-phenyl-3,4-dihydroisoquinoline-2(1H)-carboxylate (compound IV)

0.50 g (1.37 mmol) of diastereoisomeric mixture enriched with isomer (R)—((R)-quinuclidin-3-yl) 1-phenyl-3,4-dihydroisoquinoline-2(1H)-carboxylate (d.e.=24.0%) was treated with 2 mL of 37% aqueous chlorhydric acid at 20-25° C. over 16 h under stirring. The reaction was washed with 4 mL of toluene, the aqueous phase was adjusted to pH 10 with potassium carbonate and extracted twice with 2 mL of dichloromethane. After removing the solvent at reduced pressure it was obtained 0.35 g (70%) of a mixture of (S,R)—((R)-quinuclidin-3-yl) 1-phenyl-3,4-dihydroisoquinoline-2(1H)-carboxylate (d.e.=4.0%).

1. A process for preparing solifenacin or an acid addition salt thereof, comprising reacting (R)-quinuclidin-3-yl phenethylcarbamate of formula (III) with benzaldehyde: 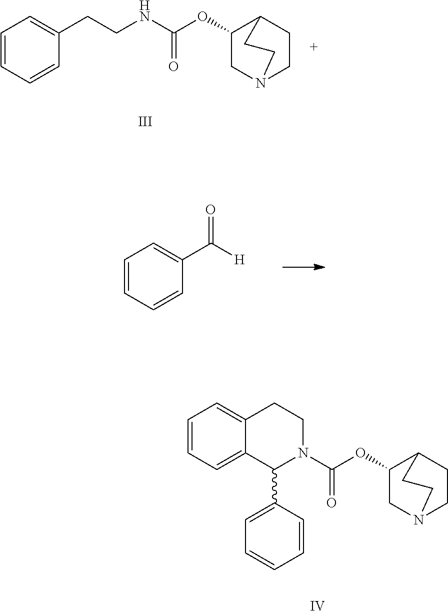
in the presence of an acid to obtain the diasteroisomeric mixture (S,R)—((R)-quinuclidin-3-yl) 1-phenyl-3,4-dihydroisoquinoline-2(1H)-carboxylate of formula (IV).

2. The process for preparing solifenacin or an acid addition salt thereof according to claim 1, wherein the acid is selected from the group consisting of organic acids, inorganic acids and Lewis acids.

3. The process for preparing solifenacin or an acid addition salt thereof, according to claim 1 wherein the acid is selected from the group consisting of sulphuric acid, acetic acid, methanesulfonic acid, boron trifluoride, titanium (IV) chloride and zinc chloride.

4. The process according to of claim 1, further comprising the separation of the diasteroisomers (S, R) and (R, R) from the diasteroisomeric mixture (S,R)—((R)-quinuclidin-3-yl) 1-phenyl-3,4-dihydroisoquinoline-2(1H)-carboxylate of formula (IV) and recovering solifenacin of formula (V) or an acid addition salt thereof.

5. The process according to claim 4, wherein separation is carried out by a fractional crystallization method comprising the following steps:
(i) treating the diasteroisomeric mixture of formula (IV) in a solvent with an organic or inorganic acid,
(ii) separating a first precipitate enriched in the addition salt of (S)—((R)-quinuclidin-3-yl) 1-phenyl-3,4-dihydroisoquinoline-2(1H)-carboxylate with said acid from the mother liquor enriched in the addition salt of (R)—((R)-quinuclidin-3-yl) 1-phenyl-3,4-dihydroisoquinoline-2(1H)-carboxylate with said acid and
(iii) recovering the addition salt of (S)—((R)-quinuclidin-3-yl) 1-phenyl-3,4-dihydroisoquinoline-2(1H)-carboxylate with said acid.

6. The process according to claim 5, wherein said acid is selected from the group consisting of succinic acid, oxalic acid and sulphuric acid.

7. The process according to claim 5, further comprising:
a) treating the mother liquor enriched in (R)—((R)-quinuclidin-3-yl) 1-phenyl-3,4-dihydroisoquinoline-2(1H)-carboxylate with a base or a second acid and
b) recovering of a diasteroisomeric mixture (S,R)—((R)-quinuclidin-3-yl) 1-phenyl-3,4-dihydroisoquinoline-2(1H)-carboxylate (IV).

8. The process according to claim 7, wherein said base or second acid is selected from the group consisting of methanesulfonic acid, sulfuric acid, aqueous concentrated clorhydric acid, potassium tert-butoxide, sodium hydride, lithium bis(trimethylsilyl)amide and potassium lithium bis(trimethylsilyl)amide.

9. The process according to claim 1, wherein compound (R)-quinuclidin-3-yl phenethylcarbamate of formula (III) is prepared by reacting 2-phenethylamine with a compound of formula (I) wherein Lv represents a leaving group, in a solvent.

10. The process according to claim 9, wherein the step of reacting 2-phenethylamine with a compound of formula (I) is performed in the presence of a base, said base being a trialkylamine.

11. The process according to claim 9, wherein compound (I) is prepared by reacting a compound of formula Lv-C(O)-Lv with 3(R)-quinuclidinol, where Lv, the same or different, represents a leaving group, in a solvent.

12. The process according to claim 1, wherein (III) is prepared by reacting a compound of formula (II) wherein Lv represents a leaving group, with 3(R)-quinuclidinol in a solvent in the presence of a base.

13. The process according to claim 12, wherein the leaving group presents the formula —OR, wherein R represents a linear or branched C1-C6 alkyl or aryl group, optionally substituted, the base is a metal alkoxide or sodium hydride.

14. The process according claim 12, wherein compound (II) is prepared by reacting 2-phenethylamine with a compound of formula Lv-C(O)-Lv where Lv, the same or different, represents a leaving group in a solvent.

15. Compound (R)-quinuclidin-3-yl phenethylcarbamate (III) 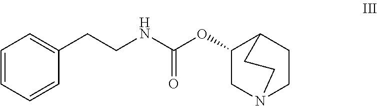 [SMILES: O=C(NCCc1ccccc1)O[C@H]1CN2CCC1CC2]
or a salt thereof.

16. A method for the transformation of (R)—((R)-quinuclidin-3-yl) 1-phenyl-3,4-dihydroisoquinoline-2(1H)-carboxylate into a diasteroisomeric mixture, (S,R)—((R)-quinuclidin-3-yl) 1-phenyl-3,4-dihydroisoquinoline-2(1H)-carboxylate (IV) which comprises the following steps:
treating (R)—((R)-quinuclidin-3-yl) 1-phenyl-3,4-dihydroisoquinoline-2(1H)-carboxylate, or a diasteroisomeric mixture enriched with (R)—((R)-quinuclidin-3-yl) 1-phenyl-3,4-dihydroisoquinoline-2(1H)-carboxylate or an acid addition salt thereof, with a strong acid, and
recovering the resulting diasteroisomeric mixture (S,R)—((R)-quinuclidin-3-yl) 1-phenyl-3,4-dihydroisoquinoline-2(1H)-carboxylate (IV).

17. The method for the transformation according to claim 16, wherein the strong acid used is methanesulfonic acid, sulfuric acid, aqueous concentrated chlorhydric acid or mixtures thereof.

18. The method for the transformation according to claim 17, wherein said acid is used in an amount of 1 or more equivalents.

19. The process according to claim 7 wherein the diasteroisomeric mixture (S,R)—((R)-quinuclidin-3-yl) 1-phenyl-3,4-dihydroisoquinoline-2(1H)-carboxylate (IV) is further submitted to the following steps of a fractional crystallization method:
(i) treating said diasteroisomeric mixture with an organic or inorganic acid;
(ii) separating a second precipitate enriched in the addition salt of (S)—((R)-quinuclidin-3-yl) 1-phenyl-3,4-dihydroisoquinoline-2(1H)-carboxylate with said acid from the mother liquor enriched in the addition salt of (R)—((R)-quinuclidin-3-yl) 1-phenyl-3,4-dihydroisoquinoline-2(1H)-carboxylate with said acid; and
(iii) recovering of the addition salt of (S)—((R)-quinuclidin-3-yl) 1-phenyl-3,4-dihydroisoquinoline-2(1H)-carboxylate with said acid.
